(19) United States
(12) Patent Application Publication
(10) Pub. No.: US 2026/0199110 A1
(43) Pub. Date: Jul. 16, 2026

(54) STENT DEPLOYMENT SYSTEM

(71) Applicant: BOSTON SCIENTIFIC SCIMED, INC., Maple Grove, MN (US)

(72) Inventors: Martyn G. Folan, Galway (IE); Louis McNern, Donegal (IE); Keith McCoy, Tuam (IE); Thomas Martin Keating, Galway (IE); Michael Walsh, Tuam (IE)

(73) Assignee: BOSTON SCIENTIFIC SCIMED, INC., Maple Grove, MN (US)

(21) Appl. No.: 19/446,356

(22) Filed: Jan. 12, 2026

Related U.S. Application Data
(60) Provisional application No. 63/744,566, filed on Jan. 13, 2025.

(51) Int. Cl. A61F 2/95

(52) U.S. Cl. CPC A61F 2/9517; A61F 2002/9511

(57) ABSTRACT

Example stent delivery systems are disclosed. An example stent delivery system includes an elongate shaft having a distal end region and a proximal end region and a deployment shaft coupled to the elongate shaft, the deployment shaft including an outer surface and a spooling region. The stent delivery system also includes an actuation member disposed along the outer surface of the deployment shaft, a stent disposed on the distal end region of the elongate shaft and a thread having a distal portion configured to wrap around at least a portion of the stent and a proximal portion coupled to the spooling region. Further, translation of the actuation member along a longitudinal axis of the deployment shaft rotates the deployment shaft and rotation of the deployment shaft unwraps the thread from the stent.

### CROSS-REFERENCE TO RELATED APPLICATIONS

The application claims the benefit of U.S. Provisional Patent Application Serial No. 63/744,566, filed on January 13, 2025, the disclosure of which is incorporated herein by reference.

### TECHNICAL FIELD

The present disclosure relates to apparatuses, systems, and methods that include constraints for selective deployment of an expandable device during device delivery.

### BACKGROUND

Implantable stents are devices that are placed in a body structure, such as a blood vessel, esophagus, trachea, biliary tract, colon, intestine, stomach or body cavity, to provide support and to maintain the structure open. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

This disclosure is directed to several alternative designs, materials, and methods of manufacturing medical device structures and assemblies, for preventing leaks after an anastomosis surgery and/or treating various gastro-intestinal, digestive, or other ailments.

An example stent delivery system includes an elongate shaft having a distal end region and a proximal end region and a deployment shaft coupled to the elongate shaft, the deployment shaft including an outer surface and a spooling region. The stent delivery system also includes an actuation member disposed along the outer surface of the deployment shaft, a stent disposed on the distal end region of the elongate shaft and a thread having a distal portion configured to wrap around at least a portion of the stent and a proximal portion coupled to the spooling region. Further, translation of the actuation member along a longitudinal axis of the deployment shaft rotates the deployment shaft and rotation of the deployment shaft unwraps the thread from the stent.

Alternatively or additionally to any of the examples above, wherein rotation of the deployment shaft retracts the thread in a proximal direction.

Alternatively or additionally to any of the examples above, wherein the thread is configured to be wound around the spooling region while the thread is retracted in the proximal direction.

Alternatively or additionally to any of the examples above, further comprising a proximal collar fixedly attached to the elongate shaft.

Alternatively or additionally to any of the examples above, further comprising a distal collar fixedly attached to the elongate shaft.

Alternatively or additionally to any of the examples above, wherein the deployment shaft is positioned between the proximal collar and the distal collar.

Alternatively or additionally to any of the examples above, wherein a portion of the elongate shaft extends through the lumen of the deployment shaft.

Alternatively or additionally to any of the examples above, wherein the elongate member includes a deployment lumen and a proximal aperture, and wherein the thread extends within at least a portion of the deployment lumen.

Alternatively or additionally to any of the examples above, wherein the distal collar includes an inner cavity, and wherein the spooling region is configured to extend into the inner cavity.

Alternatively or additionally to any of the examples above, wherein the distal collar includes an opening configured to permit the thread to pass from deployment lumen, through the proximal aperture, through the opening and into the inner cavity.

Alternatively or additionally to any of the examples above, wherein the actuation member includes a projection configured to engage a helical groove extending along a portion of the outer surface of the deployment shaft.

Alternatively or additionally to any of the examples above, further comprising a grip member attached to the proximal end region of the elongate shaft.

Alternatively or additionally to any of the examples above, wherein the stent is configured to shift from a constrained configuration to an expanded configuration when unwrapped from the thread.

Alternatively or additionally to any of the examples above, wherein the proximal end of the stent, the distal end of the stent or both the proximal and distal ends of the stent shift from the constrained configuration to the expanded configuration prior to the medial region of the stent shifting from the constrained configuration to the expanded configuration.

Another stent delivery system includes an elongate shaft having a distal end region and a proximal end region and a deployment shaft coupled to the elongate shaft, the deployment shaft including an outer surface, a spooling region and a first helical groove extending along a portion of the outer surface. The stent delivery system also includes an actuation member coupled to the deployment shaft, a stent disposed on the distal end region of the elongate shaft and a thread having a distal portion and a proximal portion, the distal portion configured to wrap around at least a portion of the stent, and the proximal portion coupled to the spooling region. Further, translation of the actuation member along a longitudinal axis of the deployment shaft is configured to rotate the deployment shaft and rotation of the deployment shaft is configured to unwrap the thread from the stent.

Alternatively or additionally to any of the examples above, further comprising a proximal collar fixedly attached to the elongate shaft.

Alternatively or additionally to any of the examples above, further comprising a distal collar fixedly attached to the elongate shaft.

Alternatively or additionally to any of the examples above, wherein the deployment shaft is positioned between the proximal collar and the distal collar.

Alternatively or additionally to any of the examples above, wherein the elongate shaft extends through the lumen of the deployment shaft.

An example method for positioning a stent at a target site includes positioning a stent delivery system adjacent a target site, the stent delivery system including an elongate shaft having a distal end region and a proximal end region and a deployment shaft coupled to the elongate shaft, the deployment shaft including an outer surface and a spooling region. The stent delivery system also includes an actuation member disposed along the outer surface of the deployment shaft, a stent disposed on the distal end region of the elongate shaft and a thread having a distal portion configured to wrap around at least a portion of the stent and a proximal portion coupled to the spooling region. The method also includes translating the actuation member along a longitudinal axis of the deployment shaft and rotating the deployment shaft, wherein the rotation of the deployment shaft is configured to release the thread from the stent.

The above summary of exemplary embodiments is not intended to describe each disclosed embodiment or every implementation of the present disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example stent delivery system;

FIG. 2 is a perspective view of the distal end region of the stent delivery system of FIG. 1;

FIG. 3 is a cross-sectional view of a portion of the example stent delivery system of FIG. 1;

FIG. 4 is a cross-sectional view of a portion of the example stent delivery system of FIG. 1;

FIGS. 5-7 illustrate a method for delivering the implant of FIG. 1;

FIG. 8 illustrates a portion of another example stent delivery system;

FIG. 9 illustrates a portion of another example stent delivery system;

FIG. 10 illustrates a portion of another example stent delivery system;

FIG. 11 illustrates a portion of another example stent delivery system;

FIG. 12 is a perspective view of a distal end region of another stent delivery system;

FIGS. 13-14 illustrate a method for delivering the implant of FIG. 12;

FIG. 15 is a perspective view of a distal end region of another stent delivery system;

FIGS. 16-17 illustrate a method for delivering the implant of FIG. 15.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may be indicative as including numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

Although some suitable dimensions, ranges, and/or values pertaining to various components, features and/or specifications are disclosed, one of the skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

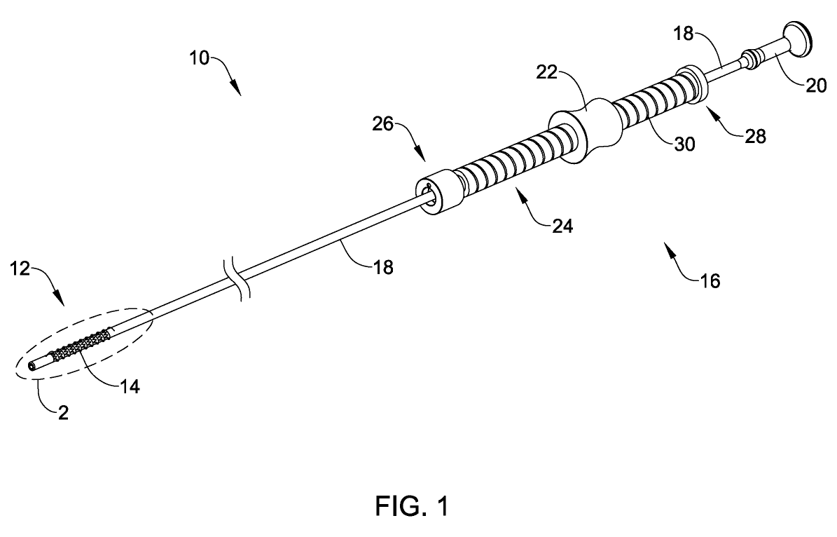

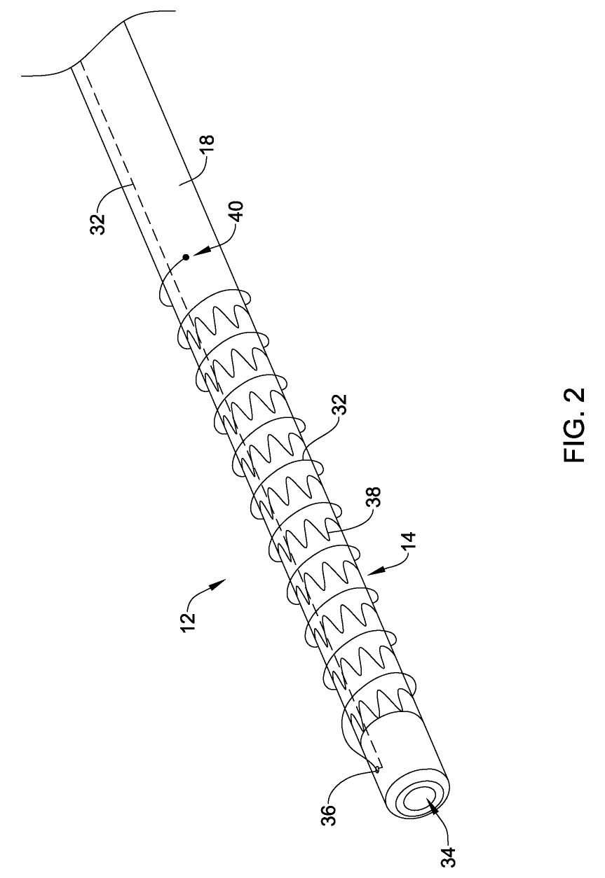

FIG. 1 is a perspective view of an illustrative delivery system 10 for delivering a stent 14 to a target region. The delivery system 10 may include an elongate shaft 18 coupled to a handle 16. The elongate shaft 18 may extend proximally from a distal end region 12 to the handle 16, whereby the handle 16 and a portion of the elongate shaft 18 may be configured to remain outside of a patient’s body. The outer shaft 18 may extend through a portion of the handle 16. Further, the outer shaft 18 may include a lumen 34 (show in FIG. 2) extending through the outer shaft 18. The lumen 34 may be configured to permit a guidewire to extend therein. The distal tip of the elongate shaft 18 may be configured to be atraumatic.

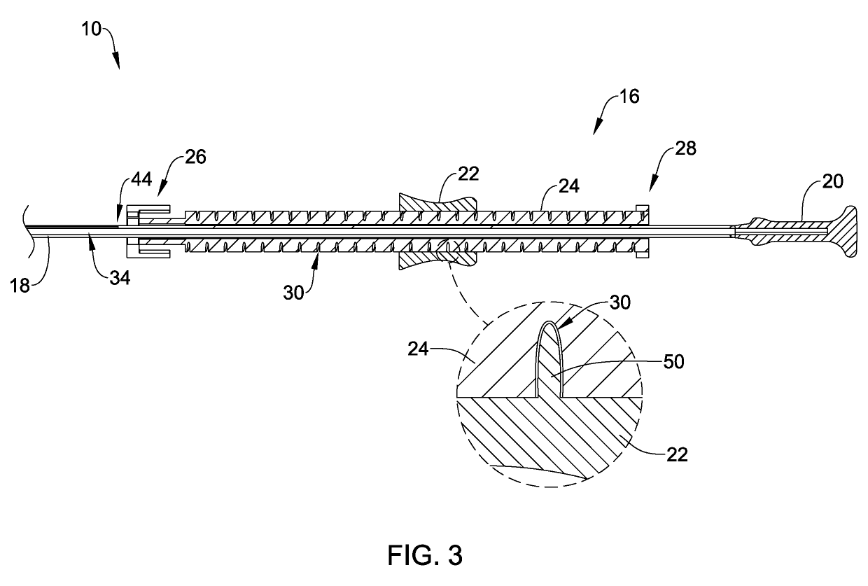

FIG. 1 further illustrates that the handle 16 may include a deployment shaft 24 coupled to the elongate shaft 18. As illustrated in FIG. 3, the deployment shaft 24 may include an inner lumen through which the elongate shaft 18 may extend. Further, FIG. 1 illustrates that the handle 16 may include a distal collar 26 coupled to a distal end region of the deployment shaft 24 and a proximal collar 28 coupled to the proximal end region of the deployment shaft 24. Both the distal collar 26 and the proximal collar 28 may each include an aperture configured to permit the elongate shaft 18 to extend therethrough. As will be described in greater detail below, the helical shaft 24 may be configured to rotate (e.g., spin) around the outer surface of the elongate shaft 18. Further, the distal collar 26 and the proximal collar 28 may be fixed to the outer shaft 18, whereby the distal collar 26 and the proximal collar 28 prevent the deployment shaft 24 from translating (e.g., sliding, moving) along the longitudinal axis of the elongate shaft 18.

FIG. 1 further illustrates the deployment shaft 24 may further include a helical groove 30 extending from a distal end region of the deployment shaft 24 to a proximal end region of the deployment shaft 24. The helical groove 30 may extend radially inward from an outer surface of the deployment shaft 24. In other words, the helical groove 30 may extend into the wall of the deployment shaft 24.

FIG. 1 further illustrates that the handle 16 may further include an actuation member 22 (e.g., bobbin) coupled to the deployment shaft 24. As will be discussed in greater detail below, the actuation member 22 may include an aperture through which the deployment shaft 24 may extend. Further, the actuation member 22 may include one or more projections extending radially inward from an inner surface of the actuation member 22, whereby the one or more projections are configured to engage the helical groove 30. The actuation member 22 may include an ergonomic shape which permits a user to easily and comfortably grasp the actuation member 22.

FIG. 1 further illustrates that the handle 16 may further include a grip member 20 fixedly attached to a proximal end region of the elongate shaft 18. The grip member 20 may be configured to include an ergonomic shape which permits a user to easily and comfortably grasp the grip member 20.

FIGS. 1-2 further illustrate that distal end region 12 of the delivery system 10 may include a stent 14 disposed along the distal end region of the elongate shaft 18. FIG. 2 illustrates the stent 14 may include an elongated tubular stent frame 38. In some examples, the stent frame 38 may be entirely, substantially or partially, covered with a polymeric covering, such as a coating (not explicitly shown). The covering may be disposed on an inner surface and/or outer surface of the stent frame 38, as desired. When so provided a polymeric covering may reduce or eliminate tissue ingrowth and/or reduce food impaction. Further, the stent 14 may include regions of differing diameters. For example, the proximal and/or distal end regions of the stent 14 may include a flared (e.g., enlarged relative to other portions of the stent 14) portion. Further, the stent frame 38 may be expandable between a radially collapsed delivery configuration and a radially expanded deployed configuration. The expanded configuration may secure the stent 14 at the desired location in a body lumen. In some cases, the stent 14 may include features (e.g., anti-migration flares, fixation spikes, fins, sutures, etc.) to prevent distal/proximal displacement and/or migration of the stent 14, after the stent 14 is positioned and expanded in the body lumen.

It can be appreciated that, in some examples, the stent frame 38 may include a woven structure, fabricated from one or more individual filaments. In some embodiments, the stent frame 38 may be braided with one filament. In other embodiments, the stent frame 38 may be braided with several filaments, as is found, for example, in the WallFlex®, WALLSTENT®, and Polyflex® stents, made and distributed by Boston Scientific. In another embodiment, the stent frame 38 may be knitted, such as the Ultraflex™ stents made by Boston Scientific. In yet another embodiment, the stent frame 38 may be of a knotted type, such the Precision Colonic™ stents made by Boston Scientific Scimed, Inc. In still another embodiment, the stent frame 38 may be laser cut, such as the EPIC™ stents made by Boston Scientific.

Additionally, it is contemplated that the stent frame 38 may be made from a number of different materials such as metals, metal alloys, shape memory alloys and/or polymers, as desired, enabling the stent 14 to be expanded into shape when accurately positioned within the body. In some instances, the material may be selected to enable the stent 14 to be removed with relative ease. For example, the stent frame 38 can be formed from alloys such as, but not limited to, nitinol and Elgiloy®. Depending the on material selected for construction, the stent 14 may be self-expanding (i.e., configured to automatically radially expand when unconstrained). In some embodiments, fibers may be used to make the stent frame 38, which may be composite fibers, for example, having an outer shell made of nitinol having a platinum core. It is further contemplated that the stent frame 38 may be formed from polymers including, but not limited to, polyethylene terephthalate (PET). In some embodiments, the stent 14 may be self-expanding while in other embodiments, the stent 14 may be expand by an expansion device (such as, but not limited to a balloon inserted within a lumen of the stent 14). As used herein the term "self-expanding" refers to the tendency of the stent to return to a preprogrammed diameter when unrestrained from an external biasing force (for example, but not limited to a delivery catheter or sheath). In some examples, the stent 14 may include a one-way valve, such as an elastomeric slit valve or duck bill valve, positioned within a lumen of the stent 14 to prevent retrograde flow of gastrointestinal fluids. A non-limiting list of materials which may be used to form the stent 14 or components thereof are disclosed herein.

It can be appreciated that when implanted in a patient, the stent 14 may exert a radially outward force to help secure the stent 14 to a body lumen. The stent 14 may be positioned in the esophagus, the gastro-esophageal junction (GEJ) region, or at or near the pylorus with the stent 14 extending through the stomach or other portions of the gastro-intestinal system. In other examples, the stent 14 may be positioned in the patient’s intestine and/or in the biliary anatomy. In further examples, the stent 14 may be used in advanced techniques including bridging procedures such as an endoscopic ultrasound-guided hepaticogastrostomy (EUS-HGS) and endoscopic ultrasound-guided gastrojejunostomy (EUS-GJ) by example.

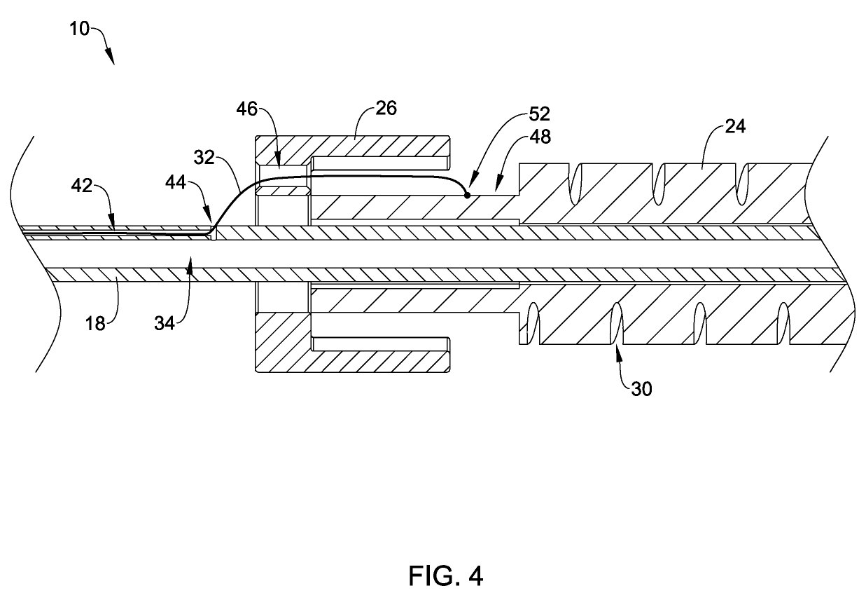

FIG. 2 further illustrates that, in some examples, the stent 14 may be held in a radially collapsed configuration through the use of a thread 32 (e.g., suture, filament, wire, etc.). The thread 32 may be any thin flexible element capable of being wrapped and unwrapped about the stent 14. FIG. 2 illustrates that a distal portion of the thread 32 may be wound or wrapped about the stent 14 whereby the distal end 40 of the thread may be releasably attached to the outer surface of the elongate shaft 18. Additionally, FIG. 2 illustrates that a portion of the thread 32 may pass through an aperture 36 positioned on the distal end region of the elongate shaft 18. As will be described in greater detail herein, a proximal portion of the thread 32 may pass through the aperture 36 and into a lumen 42 (shown in FIG. 4) of the elongate shaft 18. FIG. 2 further illustrates that the thread 32 may be wound about an outer surface of the stent 14 to apply a biasing force to the stent 14 which maintains the stent 14 in a collapsed or reduced diameter configuration. In some examples, the distal end of the thread 32 may be positioned adjacent to a proximal end region of the stent 14 and releasably attached to the outer surface of the elongate shaft 18 at an attachment point 40.

In some examples, such as that illustrated in FIG. 2, the thread 32 may be wrapped around the stent 14 in a generally helical manner. In other examples, the thread 32 may be wrapped around the stent 14 in other configurations. It can be appreciated that the spacing of adjacent windings of the thread 32 may be uniform or variable, as desired. In other words, the pitch of the windings of the thread 32 may be the same, varied, or combinations thereof, as desired. In some cases, the thread 32 may include a plurality of knots similar in form and function to those used in weft and warp knitting or crocheting patterns, which allow the thread 32 to be releasably secured about the stent 14. The knots may generally maintain the thread 32 in a desired configuration while still allowing the thread 32 to be unraveled or removed as desired. In some cases, the thread 32 may not include knots.

As discussed herein, the thread 32 may extend through an aperture 36 (e.g., skive, slot, or other opening) in the wall of the elongate shaft 18 and into the lumen 42 (shown in FIG. 4) of the elongate shaft 18. The aperture 36 may extend from an outer surface to an inner surface of the elongate shaft 18 to allow the thread 32 to extend between the exterior of the elongate shaft 18 and the inner lumen 42.

FIG. 3 illustrates a cross-sectional view of a portion of the stent delivery system 10. As discussed herein, FIG. 3 illustrates the elongate shaft 18 passing through a lumen of the deployment shaft 24. Additionally, FIG. 3 illustrates the distal collar 26 coupled to the elongate shaft 18 and positioned adjacent a distal end region of the deployment shaft 24. Further, FIG. 3 illustrates the proximal collar 28 coupled to the elongate shaft 18 and positioned adjacent a proximal end region of the deployment shaft 24. As discussed herein, it can be appreciated that both the distal collar 26 and the proximal collar 28 may be fixed (e.g., secured, attached, etc.) to the outer surface of the elongate shaft 18. Accordingly, it can be appreciated that the distal collar 26 may be configured to prevent the deployment shaft 24 from sliding distally along the longitudinal axis of the elongate shaft 18 while the proximal collar 28 may be configured to prevent the deployment shaft 24 from sliding proximally along the longitudinal axis of the elongate shaft 18. However, as discussed herein, the deployment shaft 24 may be configured to rotate (e.g., spin) around the outer surface of the elongate shaft 18 despite being prevented from sliding longitudinally along the elongate shaft 18 by the distal collar 26 and the proximal collar 28.

FIG. 3 further illustrates the grip member 20 coupled to the a proximal end region of the elongate shaft 18. Additionally, FIG. 3 illustrates the lumen 34 extending within the elongate shaft 18. The lumen 34 may extend from the distal end of the elongate shaft 18 and through the grip member 20. Accordingly, this configuration may permit a guidewire to pass through the grip member 20 and within the entire length of the elongate shaft 18. In some examples, the lumen 34 may be utilized to as a flush lumen, whereby a fluid is passed through the lumen 34 to a target region within the patient. In some examples, a fluid flush port may be attached to the grip member 20. FIG. 3 further illustrates the lumen 42 extend within the elongate shaft 18. Additionally, FIG. 3 illustrates an aperture 44 positioned adjacent to the distal collar 26. The aperture 44 may extend from an outer surface of the shaft 18 to the lumen 42. In other words, the aperture 44 may be in communication with the lumen 42.

FIG. 3 further illustrates the actuation member 22 (e.g., bobbin) coupled to the deployment shaft 24. Further, the detailed view of FIG. 3 illustrates the actuation member 22 may include one or more projections 50 extending radially inward from an inner surface of the actuation member 22, whereby the one or more projections 50 are configured to engage the helical groove 30. Accordingly, it can be appreciated from FIG. 3 that linear translation of the actuation member 22 along the longitudinal axis of the deployment shaft 24 may cause the deployment shaft 24 to spin. In other words, because the projection 50 of the actuation member 22 engages the helical groove 30 of the deployment shaft 24, a linear, distal-to-proximal pulling of the actuation member 22 along the longitudinal axis of the deployment shaft 24 will impart a force from the projection 50 onto the deployment shaft 24 via the path of the helical groove 30, thereby causing the deployment shaft 24 to spin in a clockwise direction as viewed from the grip member 20 and, conversely, a linear, proximal-to-distal pushing of the actuation member 22 along the longitudinal axis of the deployment shaft 24 will impart a force from the projection 50 onto the deployment shaft 24 via the path of the helical groove 30, thereby causing the deployment shaft 24 to spin in a counter-clockwise direction as viewed from the grip member 20.

Additionally, in yet other examples, the helical groove 30 may be configured such that a linear, distal-to-proximal pulling of the actuation member 22 along the longitudinal axis of the deployment shaft 24 will impart a force from the projection 50 onto the deployment shaft 24 via the path of the helical groove 30, thereby causing the deployment shaft 24 to spin in a counter-clockwise direction as viewed from the grip member 20 and, conversely, a proximal-to-distal pushing of the actuation member 22 along the longitudinal axis of the deployment shaft 24 will impart a force from the projection 50 onto the deployment shaft 24 via the path of the helical groove 30, thereby causing the deployment shaft 24 to spin in a clockwise direction as viewed from the grip member 20.

FIG. 4 illustrates a cross-sectional view of a portion of the delivery system 10. FIG. 4 illustrates that the distal end region of the deployment shaft 24 may include a spooling region 48 extending distally away from the helical groove 30 of the deployment shaft 24. The spooling region 48 may include a smooth outer surface configured to extend into an inner cavity (e.g., recess, etc.) of the distal collar 26. FIG. 4 further illustrates that the distal end of the spooling region may engage and/or contact an inner surface of the distal collar 26. As discussed herein, the distal collar 26 may be configured to prevent the deployment shaft 24 from sliding along the elongate shaft 18 in a distal direction. In some examples, the collar 26 may be configured to fully encapsulate and/or cover the spooling region 48. In this configuration, the spooling region 48 may not be visible to the user.

FIG. 4 further illustrates the thread 32 extending within the lumen 42 of the elongate shaft 18. As discussed herein, FIG. 4 illustrates that the thread 32 may exit the lumen through the aperture 44. Additionally, FIG. 4 illustrates that that the thread 32 may pass into through an opening 46 (e.g., eyehole, eyelet, aperture, etc.) extending through a wall of the distal collar 26. It can be appreciated that a proximal end of the thread 32 may pass through the opening 46 and fixedly attach to the outer surface of the spooling region 48 at an attachment point 52. FIG. 4 further illustrates the lumen (e.g., guidewire lumen, flushing lumen, etc.) extending within the elongate member 18.

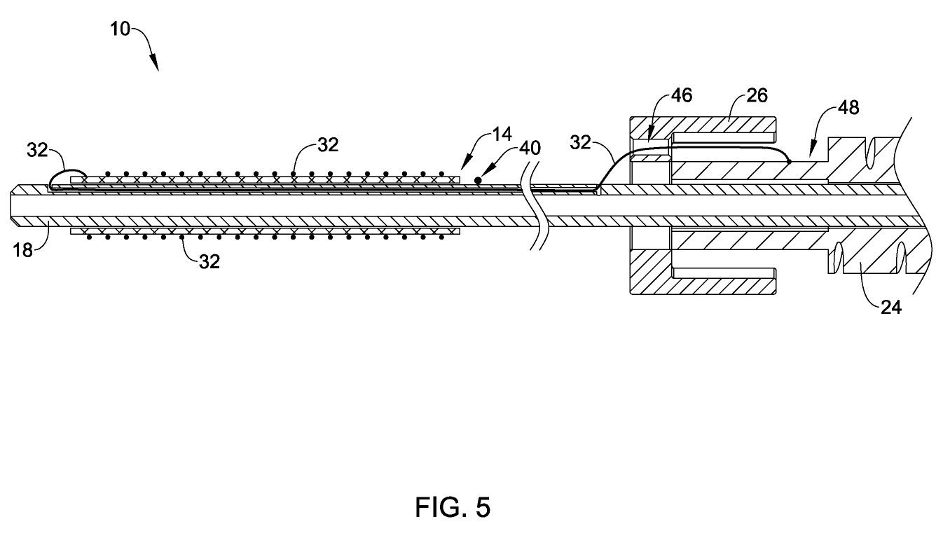

FIGS. 5-7 illustrate a method of deploying the example stent 14 to a body lumen using the delivery device 10 of FIG. 1.

FIG. 5 illustrates a cross-sectional view of a portion of the delivery system 10. FIG. 5 illustrates the stent 14 positioned on the distal end region of the elongate member 18 in a collapsed or delivery configuration. FIG. 5 illustrates that the stent 14 may be maintained in the collapsed or delivery configuration via the thread 32 being wrapped around the outer surface of the stent 14, whereby the distal end of the thread 32 is releasably attached to the outer surface of the elongate shaft 18 at an attachment point 40. Further, as discussed herein, FIG. 5 illustrates the thread 32 passing into the aperture 36 (shown in FIG. 2), extending proximally within the lumen 42 (shown in FIG. 4), passing through the aperture 44 (shown in FIG. 4), passing through the opening 46 and attached to the outer surface of the spooling region 48. The delivery device 10 may be advanced to a target site with or without the use of a guidewire.

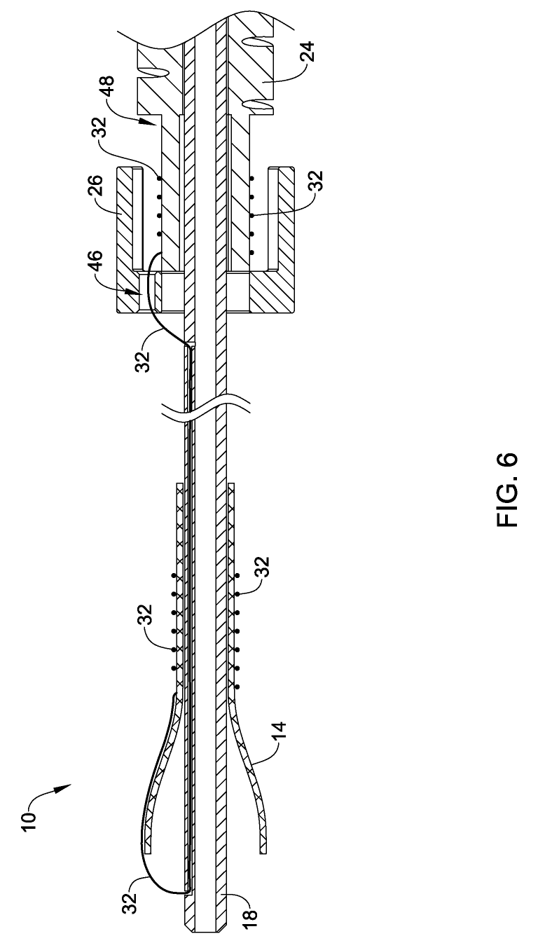

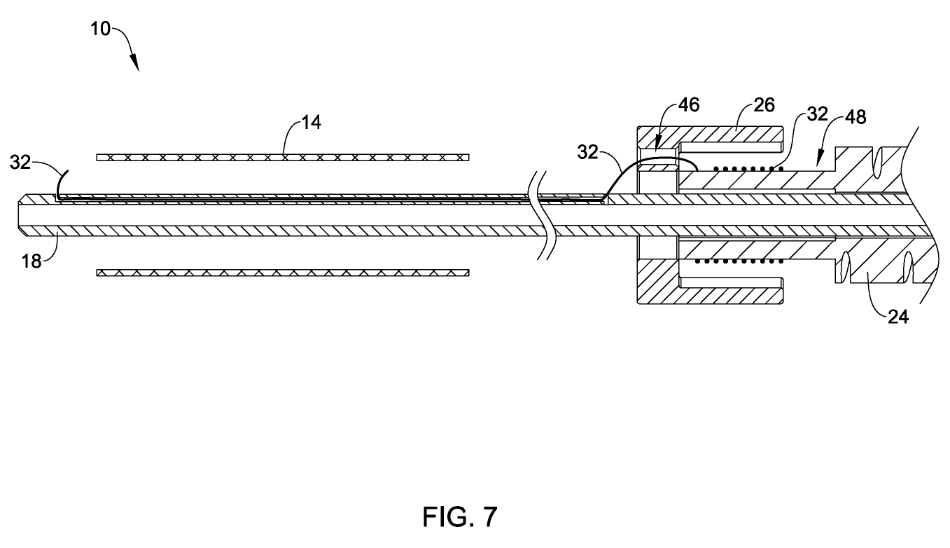

FIGS. 6-7 illustrate that after the stent 14 is positioned adjacent to a target region, the restraining force of the thread 32 maintaining the stent 14 in the radially collapsed configuration may be removed to deploy the stent 14. In some examples, a first step in deploying the stent 14 may include an operator grasping and/or anchoring the grip member 20 (shown in FIG. 3) with one hand, while gripping and linearly translating the actuation member 22 (shown in FIG. 3) with the opposite hand. As described herein, FIG. 6 illustrates that the distal-to-proximal linear translation of the actuation member 22 along the longitudinal axis of the deployment shaft 24 may rotate (e.g., spin) the spooling region 48 of the deployment shaft 24. Further, FIG. 6 illustrates that the rotation of the spooling region 48 may begin to wind the thread 32 (the distal end of which is attached to the spooling region as described with respect to FIG. 5) onto the spooling region 48. Further, the winding of the thread 32 onto the spooling region 48 may pull the thread 32 in a proximal direction through the aperture 44 (shown in FIG. 4), the lumen 42 (shown in FIG. 4) and the aperture 36 (shown in FIG. 2). Additionally, the proximal retraction of the thread 32 may further release the distal end of the thread 32 from the outer surface of the elongate member 18. As the distal end of the thread 32 is released from the outer surface of the elongate member 18, the thread 32 may begin to unravel, thereby permitting the distal end region of the stent 14 to expand into its unbiased or deployed configuration. In the embodiments shown in FIGS. 1-7, the thread 32 may be wrapped or wound such that the portion of the thread 32 disposed over the distal region of the stent 14 is removed or unraveled first, thereby permitting the distal end region of the stent 14 to expand prior to the proximal end region (as shown in FIG. 6). However, it is contemplated that the thread 32 may be wrapped or wound such that the thread 32 disposed over the proximal region of the stent 14 is removed or unraveled first, thereby permitting the proximal end region of the stent 14 to expand prior to the distal end region.

FIG. 7 illustrates that the linear translation of the actuation member 22 may continue to wind and proximally withdraw the thread 32 until the thread 32 has been completely unraveled, thereby releasing and deploying the entire length of the stent 14. It is contemplated that an operator may continue to proximally withdraw the thread 32 until the distal end of the thread 32 has been withdrawn into the lumen 42 (shown in FIG. 4), although this is not required. After the stent 14 is deployed, the delivery device 10 may then be removed from the patient.

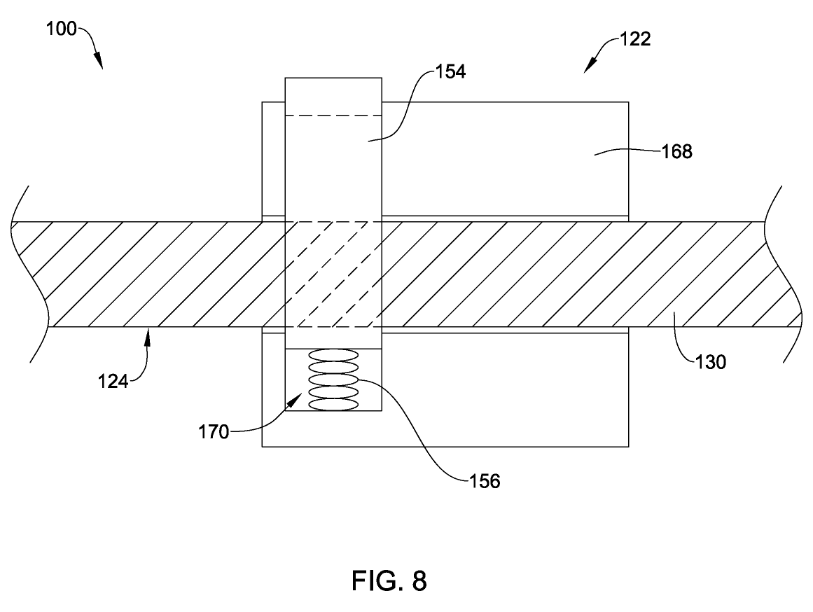

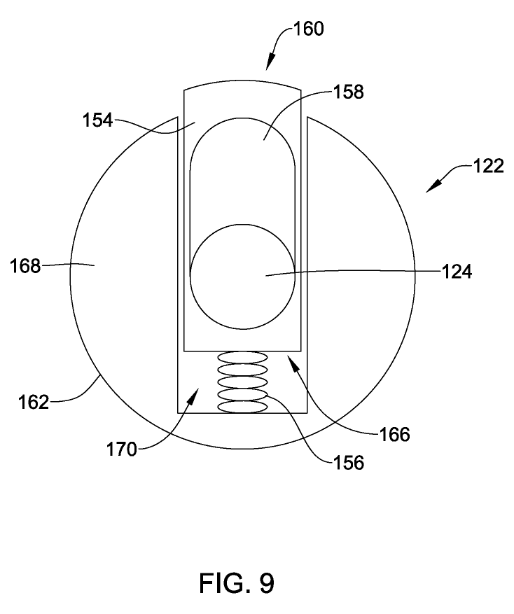

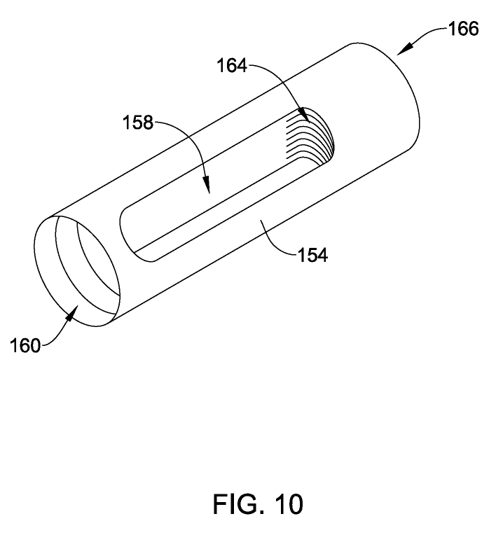

FIGS. 8-9 illustrates a portion of an example stent delivery system 100. The stent delivery system 100 may be similar in form and function to the stent delivery system 10 described herein. It can be appreciated that FIG. 8 illustrates a side view of a portion of a delivery shaft 124 (similar in form and function to the delivery shaft 24 described herein). The delivery shaft 124 includes a helical groove 130 (similar in form and function to the helical groove 30 described herein). Further, FIG. 8 illustrates the delivery shaft 124 extending through an actuation member assembly 122. The actuation member assembly 122 may include an actuation button 154 (shown in isolation in FIG. 10) positioned within a cavity 170 of an actuation housing 168 (the actuation housing 168 may be similar in form to the actuation member 22 described herein). Accordingly, FIG. 8 illustrates the delivery shaft 124 extending through both the actuation button 154 and the actuation housing 168. FIG. 8 further illustrates that the actuation member assembly 122 may further include a spring 156 having a first end which may engage the actuation housing 168 and a second end which may engage a bottom surface 166 (shown in FIG. 10) of the actuation button 154.

FIG. 9 illustrates a front view of the portion of the example stent delivery system 10 shown in FIG. 8, including the actuation button 154 positioned within the cavity 170 of the actuation housing 122. FIG. 9 further illustrates a first end of the spring 156 engaging the bottom surface 166 of the actuation button 154 and a second end of the spring 156 engaging a surface of the cavity 170 of the actuation housing 122. It can be appreciated that the spring 156 may be configured to impart an upward force on the actuation button 154. It can be further appreciated that the upward force imparted on the actuation button 154 may push the actuation button 154 upward and partially out of the cavity 170. For example, FIG. 9 illustrates a top surface 160 of the actuation button 154 extending away from the outer surface 162 of the actuation housing 168. However, it can be appreciated that the delivery shaft 124 (via extending through both the actuation button 154 and the actuation housing 168) may limit the extent to which spring 156 may push the actuation button 154 upward and partially out of the cavity 170.

As discussed herein, FIG. 9 illustrates the deployment shaft 124 extending through an aperture 158 of the actuation button. Accordingly, FIG. 9 further illustrates that the helical groove 130 (shown in FIG. 8) of the deployment shaft 124 may engage and mate with a threaded region 164 of the actuation button 154. Therefore, it can be appreciated that the deployment shaft 124 may be prevented from linear translation along the longitudinal axis of the deployment shaft 124 when the helical groove 130 of the deployment shaft 124 is engaged with the threaded region 164 of the actuation button 154. However, depressing the actuation button 154 (via pressing down on the top surface 160 of the actuation button 154) may permit the actuation button to 154 compress the spring 156 and slide down into the cavity 170 of the actuation housing 168, thereby disengaging the helical groove 130 of the deployment shaft 124 from the threaded region 164 of the actuation button 154 and permitting the actuation assembly 122 (including the actuation housing 168, the actuation button 154 and the spring 156) to translate linearly translation along the longitudinal axis of the deployment shaft 124. It can be appreciated that linear translation of the actuation assembly 122 along the helical groove 130 may rotate the deployment shaft 124 and deploy the stent 14, as discussed herein.

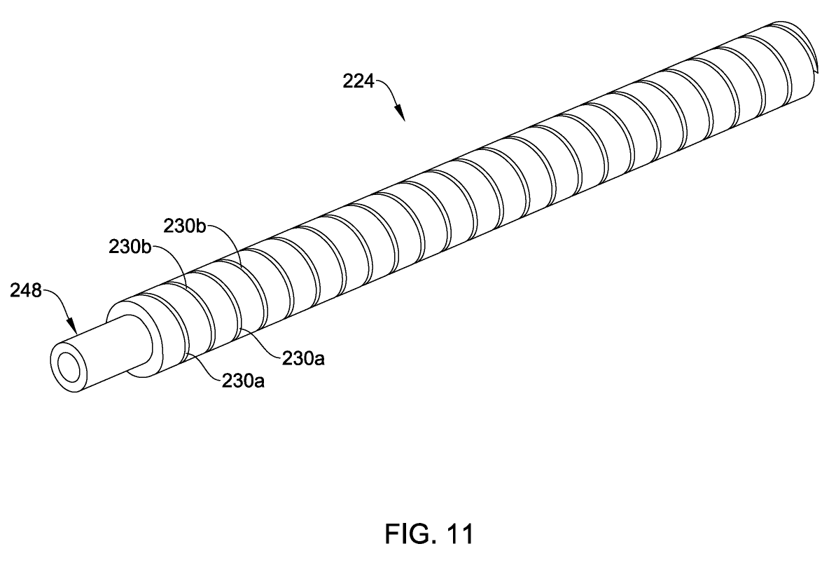

FIG. 11 illustrates another example deployment shaft 224. The deployment shaft 224 may be similar in form and function to the deployment shaft 24 described herein. The deployment shaft 224 may be used with stent delivery systems similar in form and function to the stent delivery system 10 described herein.

FIG. 11 illustrates that the deployment shaft 224 may include a “multi-groove” (e.g., multi-start thread) configuration having a first groove 230a intertwined with a second groove 230b. As illustrated in FIG. 11, each of the first groove 230a and the second groove 230b may extend parallel to one another along the length (not including the spooling region 248 shown in FIG. 11) of the deployment shaft 224. FIG. 11 further illustrates that the deployment shaft 224 may include a spooling region 248 which may be similar in form and function to the spooling region 48 described herein.

As illustrated in FIG. 1 a single groove (e.g., single start thread) 30 may include a single thread. Accordingly, the thread pitch may be equal to the linear travel per revolution actuated by the actuation member 22. As illustrated in FIG. 11, the first groove 230a and second groove 230b allows that one revolution of the deployment shaft 224 actuated by the actuation member 22 would achieve a two-fold stroke (e.g., linear travel = 2 x Pitch). The start of the grooves 230a and 230b may be offset by 180 degrees (e.g., opposite each other). The use of multiple grooves (multi-start threads) results in the clearance between the tooth flanks being reduced, which may permit the entire actuation system to be more precise.

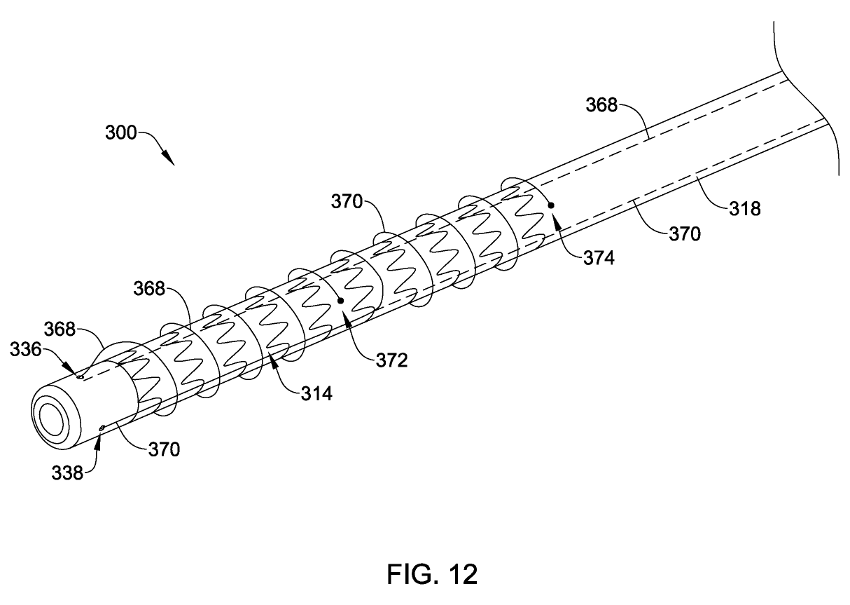

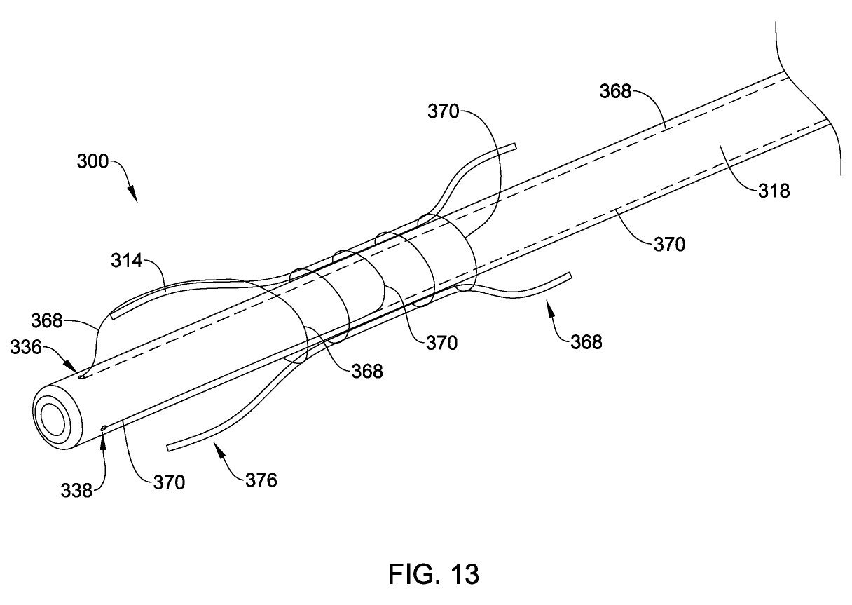

FIGS. 12-13 illustrates an example stent deployment system 300 which may be similar in form and function to components of the stent deployment system 10 described herein.

FIG. 12 illustrates a stent 314 positioned along a distal end region of an elongate member 318. FIG. 12 illustrates the stent 314 being maintained in a radially collapsed configuration via a first thread 368 (e.g., suture, filament, wire, etc.) being wrapped around the distal end region of the stent 314 and a second thread 370 (e.g., suture, filament, wire, etc.) being wrapped around a proximal end region of the stent 314. FIG. 12 further illustrates the first thread 368 extending within an inner lumen of a multi-lumen elongate shaft 318, whereby the first thread 368 passes through an aperture 336 positioned along the distal end region of the elongate member 318 and continues to wrap around the distal end region of the stent 314. FIG. 12 illustrates the distal end of the first thread 368 being releasably attached to the outer surface of the elongate shaft 318 at an attachment point 372. FIG. 12 further illustrates the second thread 370 extending within another inner lumen of a multi-lumen elongate shaft 318, whereby the second thread 370 passes through an aperture 338 positioned along the distal end region of the elongate member 318 and continues to wrap around the proximal end region of the stent 314. FIG. 12 illustrates the distal end of the second thread 370 being releasably attached to the outer surface of the elongate shaft 318 at an attachment point 374.

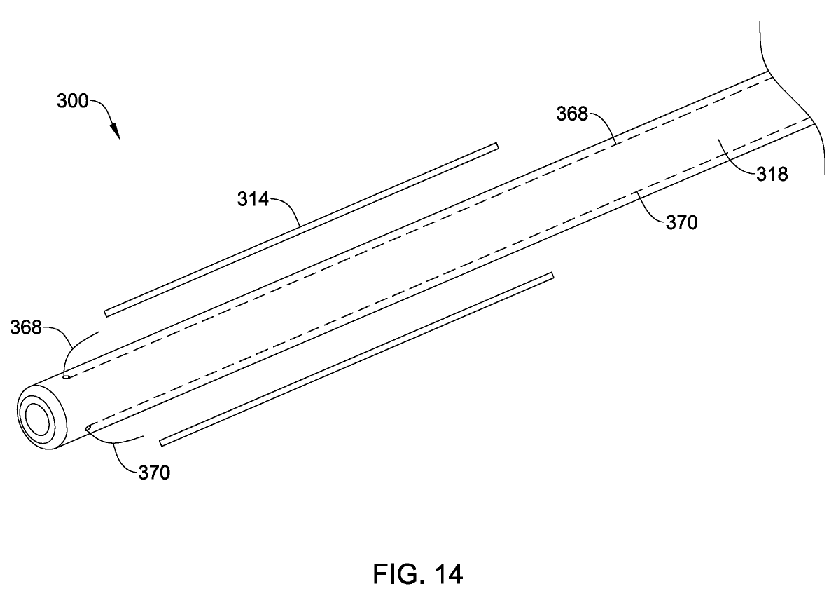

FIG. 13 illustrates that after the stent 314 is positioned adjacent to a target region, the restraining force of the first thread 368 and the second thread 370 maintaining the stent 14 in the radially collapsed configuration may be removed to deploy the stent 14. As described herein, rotation of a deployment shaft (e.g., deployment shaft 24) via linear translation of an actuation member (e.g., actuation member 22 or actuation member assembly 122 described herein) may begin to retract the first thread 368 and the second thread 370 into the inner lumens of the elongate shaft 318 through the apertures 336, 338 respectively. Additionally, the proximal retraction of the thread 368 may further release the distal end of the thread 368 from the outer surface of the elongate member 318. As the distal end of the thread 368 is released from the outer surface of the elongate member 318, the thread 368 may begin to unravel, thereby permitting the distal end region of the stent 314 to expand into its unbiased or deployed configuration. Further, the proximal retraction of the thread 370 may further release the distal end of the thread 370 from the outer surface of the elongate member 318. As the distal end of the thread 370 is released from the outer surface of the elongate member 318, the thread 370 may begin to unravel, thereby permitting the proximal end region of the stent 314 to expand into its unbiased or deployed configuration. FIG. 14 illustrates the stent 314 after both threads 368, 370 have been unwound and the entire length of the stent 314 has been fully deployed. In the embodiments shown in FIGS. 12-13, the threads 368, 370 may be wrapped or wound such that the portions of the threads 368, 370 disposed over the distal region and the proximal end region of the stent 314 are removed or unraveled first, thereby permitting the distal end region and the proximal end region of the stent 314 to expand prior to the medial region of the stent 314.

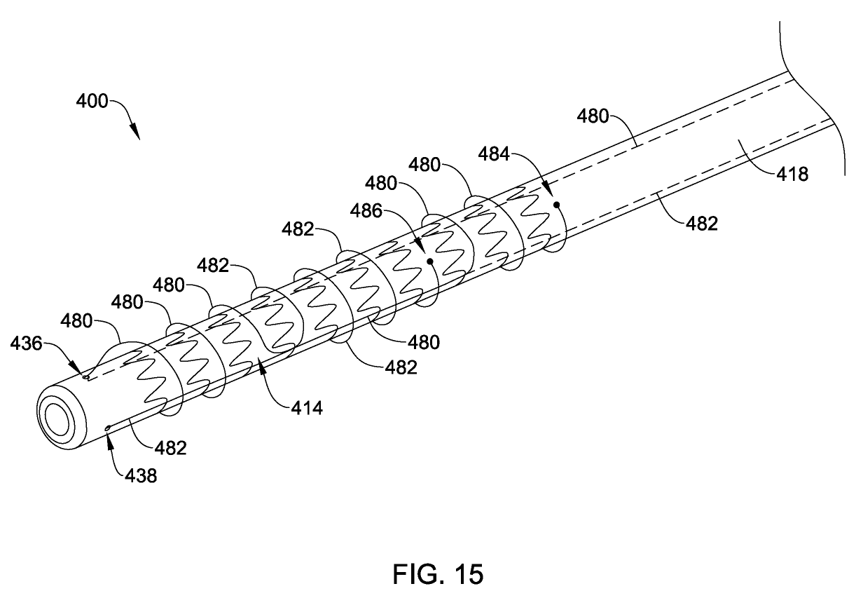

FIGS. 15-17 illustrate an example stent deployment system 400 which may be similar in form and function to components of the stent deployment system 10 described herein.

FIG. 15 illustrates a stent 414 positioned along a distal end region of an elongate member 418. FIG. 15 illustrates the stent 414 being maintained in a radially collapsed configuration via a first thread 480 (e.g., suture, filament, wire, etc.) being wrapped around both the distal and proximal end regions of the stent 414 and a second thread 482 (e.g., suture, filament, wire, etc.) being wrapped around a medial region of the stent 414. FIG. 15 further illustrates the first thread 480 extending within a first inner lumen of a multi-lumen elongate shaft 418, whereby the first thread 480 passes through an aperture 436 positioned along the distal end region of the elongate member 418 and wraps around both the distal end region and the proximal end region of the stent 414. FIG. 15 illustrates the distal end of the first thread 480 being releasably attached to the outer surface of the elongate shaft 418 at an attachment point 484. FIG. 15 further illustrates the second thread 482 extending within a second inner lumen of a multi-lumen elongate shaft 418, whereby the second thread 482 passes through an aperture 438 positioned along the distal end region of the elongate member 418 and wraps around the medial region of the stent 414. FIG. 15 illustrates the distal end of the second thread 482 being releasably attached to the outer surface of the elongate shaft 418 at an attachment point 486.

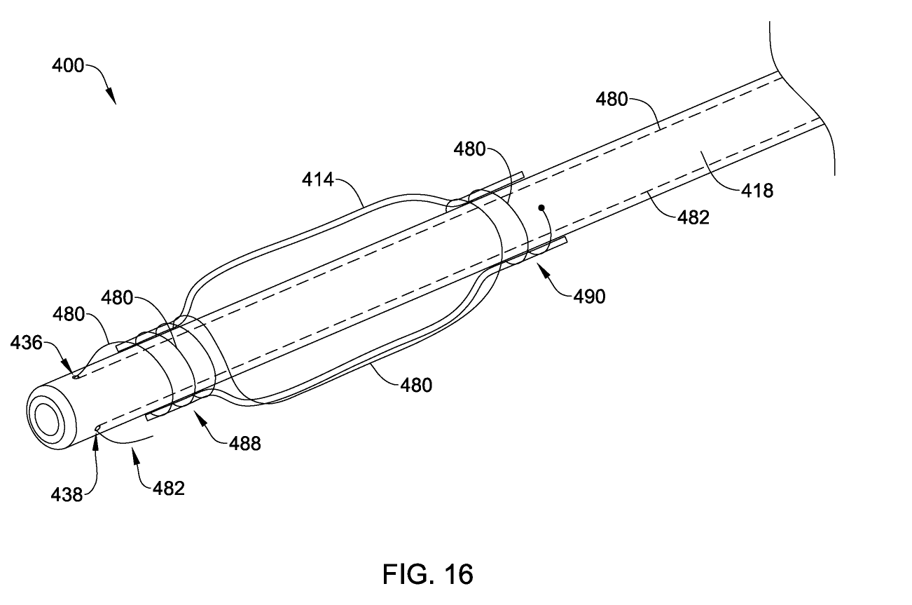

FIG. 16 illustrates that after the stent 414 is positioned adjacent to a target region, the restraining forces of the first thread 480 and the second thread 482 maintaining the stent 414 in the radially collapsed configuration may be removed to deploy the stent 414. As described herein, rotation of a deployment shaft (e.g., deployment shaft 24) via linear translation of an actuation member (e.g., actuation member 22 or actuation member assembly 122 described herein) may begin to retract the first thread 480 and the second thread 482 into the inner lumens of the elongate shaft 418 through the apertures 436, 438 respectively. However, it can be appreciated that in some examples, such as the example described with respect to FIGS. 15-17, the first thread 480 may include extra slack (e.g., length of thread 480) compared to the second thread 482. In some examples this extra slack (e.g., length of thread 480) may be stored within the inner lumen of the elongate member 418 and may be configured to delay the deployment of the medial region of the stent 414 relative to the distal and proximal end regions of the stent 414. In other words, the first thread 480 may be longer than the second thread 482, whereby the extra length of the thread 480 may take additional time to be wound up relative to the second thread 482, thereby delaying the release of the thread 482 from the distal and proximal end regions of the stent 414 relative to the release of the medial region of the stent 414. Accordingly, the proximal retraction of the thread 482 may release the distal end of the thread 482 from the outer surface of the elongate member 418 prior to the release of the distal end of the thread 480 from the outer surface of the elongate member 418. As the distal end of the thread 482 is released from the outer surface of the elongate member 418, the thread 482 may begin to unravel, thereby permitting the medial region of the stent 414 to expand into its unbiased or deployed configuration prior to the expansion of the distal and proximal end regions of the stent 414.

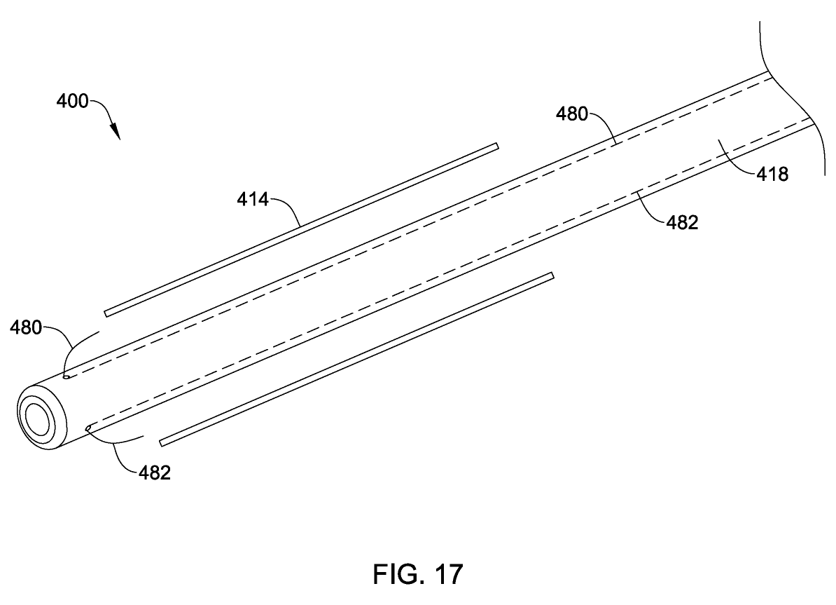

Further, after continued rotation of a deployment shaft (e.g., deployment shaft 24) and the eventual uptake of the additional length of the first thread 480, the continued proximal retraction of the first thread 480 may release the distal end of the thread 480 from the outer surface of the elongate member 418. As the distal end of the thread 480 is released from the outer surface of the elongate member 418, the thread 480 may begin to unravel, thereby permitting the distal and proximal end regions of the stent 414 to expand into its unbiased or deployed configuration. FIG. 17 illustrates the stent 414 after both threads 480, 482 have been unwound and the entire length of the stent 414 has been fully deployed. In the embodiments shown in FIGS. 15-17, the threads 480, 482 may be wrapped or wound such that the portions of the thread 482 disposed over the medial region of the stent 414 are unraveled first relative to the portions of the thread 480 disposed over the distal and proximal end regions of the stent 414, thereby permitting the medial region of the stent 414 to expand prior to the distal and proximal end regions of the stent 414.

Any of the components of the system 10 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "superelastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Patent Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, any of the components of the system 10 may also be doped with, made of, or otherwise include a radiopaque material. Radiopaque materials are generally understood to be materials which are opaque to RF energy in the wavelength range spanning x-ray to gamma-ray (at thicknesses of <0.005”). These materials are capable of producing a relatively dark image on a fluoroscopy screen relative to the light image that non-radiopaque materials such as tissue produce. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of any of the components of the system 10 to achieve the same result.

In some embodiments, a degree of Magnetic Resonance Imaging (MRI) compatibility may be imparted into any of the components of the system 10. For example, any of the components of the system 10 may be made of a material that does not substantially distort the image and create substantial artifacts (i.e., gaps in the image). Certain ferromagnetic materials, for example, may not be suitable because they may create artifacts in an MRI image. Any of the components of the system 10 may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for any of the components of the system 10 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Those skilled in the art will appreciate that the different embodiments of the stent delivery system 10 and variations thereof described herein, their mode of operation, etc., are merely representative of the environment in which the present disclosure operates. Accordingly, a variety of alternatively shaped collaborating components may also be used as a substitutive for the purpose of engaging, steering and locating the stent at a desired target site, thus, not limiting the scope of the present disclosure. Further, the disclosed stents may be adequately stretchable, extendable, and retractable, allowing for its flexible deployment. More particularly, the configurations described here may be applicable for other medical applications as well, and accordingly, a variety of other medical devices may be used in combination with the stent.

Further, while stents disclosed herein are generally described along with an exemplary rigid and flexible region(s), a variety of other configurations and arrangements may also be contemplated and conceived as well. In addition, the operations, devices, and components described herein may be equally applicable for other purposes where a component is required to be positioned in places where a leakage needs to be avoided or other treatments are desired. Embodiments of the present disclosure are thus applicable to medical and/or non-medical environments. Further, certain aspects of the aforementioned embodiments may be selectively used in collaboration, or removed, during practice, without departing from the scope of the disclosed embodiments.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A stent delivery system, comprising:
an elongate shaft having a distal end region and a proximal end region;
a deployment shaft coupled to the elongate shaft, the deployment shaft including an outer surface and a spooling region;
an actuation member disposed along the outer surface of the deployment shaft;
a stent disposed on the distal end region of the elongate shaft; and
a thread having a distal portion configured to wrap around at least a portion of the stent and a proximal portion coupled to the spooling region;
wherein translation of the actuation member along a longitudinal axis of the deployment shaft rotates the deployment shaft;
wherein rotation of the deployment shaft unwraps the thread from the stent.

2. The stent delivery system of claim 1, wherein rotation of the deployment shaft retracts the thread in a proximal direction.

3. The stent delivery system of claim 1, wherein the thread is configured to be wound around the spooling region while the thread is retracted in the proximal direction.

4. The stent delivery system of claim 1, further comprising a proximal collar fixedly attached to the elongate shaft.

5. The stent delivery system of claim 4, further comprising a distal collar fixedly attached to the elongate shaft.

6. The stent delivery system of claim 5, wherein the deployment shaft is positioned between the proximal collar and the distal collar.

7. The stent delivery system of claim 6, wherein a portion of the elongate shaft extends through the lumen of the deployment shaft.

8. The stent delivery system of claim 7, wherein the elongate member includes a deployment lumen and a proximal aperture, and wherein the thread extends within at least a portion of the deployment lumen.

9. The stent delivery system of claim 8, wherein the distal collar includes an inner cavity, and wherein the spooling region is configured to extend into the inner cavity.

10. The stent delivery system of claim 9, wherein the distal collar includes an opening configured to permit the thread to pass from deployment lumen, through the proximal aperture, through the opening and into the inner cavity.

11. The stent delivery system of claim 1, wherein the actuation member includes a projection configured to engage a helical groove extending along a portion of the outer surface of the deployment shaft.

12. The stent delivery system of claim 1, further comprising a grip member attached to the proximal end region of the elongate shaft.

13. The stent delivery system of claim 2, wherein the stent is configured to shift from a constrained configuration to an expanded configuration when unwrapped from the thread.

14. The stent delivery system of claim 13, wherein the proximal end of the stent, the distal end of the stent or both the proximal and distal ends of the stent shift from the constrained configuration to the expanded configuration prior to the medial region of the stent shifting from the constrained configuration to the expanded configuration.

15. A stent delivery system, comprising:
an elongate shaft having a distal end region and a proximal end region;
a deployment shaft coupled to the elongate shaft, the deployment shaft including an outer surface, a spooling region and a first helical groove extending along a portion of the outer surface;
an actuation member coupled to the deployment shaft;
a stent disposed on the distal end region of the elongate shaft; and
a thread having a distal portion and a proximal portion, the distal portion configured to wrap around at least a portion of the stent, and the proximal portion coupled to the spooling region;
wherein translation of the actuation member along a longitudinal axis of the deployment shaft is configured to rotate the deployment shaft;
wherein rotation of the deployment shaft is configured to unwrap the thread from the stent.

16. The stent delivery system of claim 15, further comprising a proximal collar fixedly attached to the elongate shaft.

17. The stent delivery system of claim 16, further comprising a distal collar fixedly attached to the elongate shaft.

18. The stent delivery system of claim 17, wherein the deployment shaft is positioned between the proximal collar and the distal collar.

19. The stent delivery system of claim 18, wherein the elongate shaft extends through the lumen of the deployment shaft.

20. A method for positioning a stent at a target site, the method comprising:
positioning a stent delivery system adjacent a target site, the stent delivery system including:
an elongate shaft having a distal end region and a proximal end region;
a deployment shaft coupled to the elongate shaft, the deployment shaft including an outer surface and a spooling region;
an actuation member disposed along the outer surface of the deployment shaft;
a stent disposed on the distal end region of the elongate shaft; and
a thread having a distal portion configured to wrap around at least a portion of the stent and a proximal portion coupled to the spooling region;
translating the actuation member along a longitudinal axis of the deployment shaft; and
rotating the deployment shaft, wherein the rotation of the deployment shaft is configured to release the thread from the stent.
